(19) United States
(12) Patent Application Publication
(10) Pub. No.: US 2015/0093446 A1
(43) Pub. Date: Apr. 2, 2015

(54) Exfoliating Body Scrub Composition

(71) Applicant: Aleksandra Muller, Pasadena, CA (US)

(72) Inventor: Aleksandra Muller, Pasadena, CA (US)

(21) Appl. No.: 14/043,357

(22) Filed: Oct. 1, 2013

(51) Int. Cl. A61K 8/97; A61Q 19/00; A61Q 19/10; A61K 8/64; A61K 8/98; A61K 8/92

(52) U.S. Cl. CPC A61K 8/97; A61K 8/986; A61K 8/922; A61Q 19/10; A61K 8/64; A61Q 19/00; A61K 2800/72; A61K 2800/805; A61K 2800/75; A61K 2800/59; A61K 2800/30
USPC 424/535; 424/750

(57) ABSTRACT

An exfoliating body scrub composition is made from natural ingredients. The body scrub composition comprises ground coffee; ground whole oats; dry milk; from about 0.5% by weight to about 5% by weight of soy flour, based on the total weight of the coffee; oats; dry milk; and soy flour. The body scrub composition further comprises an optional carrier, such as an aqueous carrier, a moisturizing nonaqueous carrier, or a solid carrier. The body scrub composition may be made by combining solid ingredients comprising ground coffee, ground whole oats, dry milk, and from about 0.5% by weight to about 5% by weight of soy flour; and mixing the solid ingredients to produce a uniform powdered mixture. The uniform powdered mixture is optionally mixed with a carrier.

### BACKGROUND OF THE INVENTION

1. Field of the Invention

This invention relates generally to an exfoliating body scrub composition comprising natural ingredients.

2. Description of Related Art

Exfoliating is an important part of skin care. Exfoliating serves to remove dead skin cells and remove oil and dirt from skin pores.

In general, there are two types of exfoliants in wide use. These include physical exfoliants, which contain abrasives which scrub away dead skin cells. Abrasives may include polymeric beads, such as polyurethane; granulated sugar; ground coffee; ground oatmeal; and ground walnut shells. However, some abrasives can be too harsh, and scratch the skin.

Alternatively, a chemical exfoliant may be used. Typically, a chemical exfoliant includes one or more hydroxy acids, such as glycolic acid, citric acid, lactic acid, fruit enzymes, and salicylic acid. For someone with very dry or sensitive skin, hydroxy acids can be too harsh and irritate the skin.

Accordingly, there is a need for improved exfoliating facial and body scrubs which are safe, effective, and gentle to the skin.

Additionally, there is a for a body scrub which will not cause allergic reactions in susceptible individuals. Hydrolyzed wheat protein is a wheat-derived moisturizing ingredient that is common in cosmetics, including soap, shampoos, and body washes and scrubs. Individuals with allergies to wheat-based substances, including wheat gluten, should avoid wheat-based ingredients in cosmetics, as using products with hydrolyzed wheat protein as an ingredient can cause a severe allergic reaction, including rashes, contact dermatitis, and anaphylactic shock, after cosmetics or cleansers that contain wheat products and/or hydrolyzed wheat protein. Exposure to soaps containing hydrolyzed wheat protein or other wheat-based ingredients can trigger an allergy to wheat. Certain other grains containing gluten should also be avoided in cosmetics, including rye, barley and triticale. Oats contain a type of gluten called avenin, which lacks many of the prolamines found in wheat gluten. Accordingly, oats may be used in place of wheat in some foods and cosmetics.

Corn flour contains no gluten; however, corn also contains proteins which can trigger allergic reactions in susceptible individuals. Accordingly, corn flour and corn meal are not suitable substitutes for wheat products in cosmetics.

### SUMMARY OF THE INVENTION

In light of the present need for improved exfoliating facial and body scrubs, a brief summary of various exemplary embodiments is presented. Some simplifications and omissions may be made in the following summary, which is intended to highlight and introduce some aspects of the various exemplary embodiments, but not to limit the scope of the invention. Detailed descriptions of a preferred exemplary embodiment adequate to allow those of ordinary skill in the art to make and use the inventive concepts will follow in later sections.

The current invention relates to an exfoliating facial and body scrub which combines the effects of a physical exfoliant and a chemical exfoliant. The exfoliating facial and body scrub contains ground coffee and ground oatmeal, which act as abrasives and create a physical exfoliating action. The exfoliating facial and body scrub also contains dry milk, which contains lactic acid, which is a hydroxy acid and creates a chemical exfoliating action. The exfoliating facial and body scrub also contains soy flour. Soy flour and dry milk contain proteins which act as emulsifiers.

### DETAILED DESCRIPTION

Various exemplary embodiments disclosed herein relate to a body scrub composition, comprising a mixture of ground coffee; ground whole oats; dry milk; and from about 0.5% by weight to about 5% by weight of soy flour, based on the total weight of the coffee, oats, dry milk, and soy flour. In various embodiments, the body scrub composition may contain an optional carrier. In various embodiments, the body scrub composition comprises low fat or nonfat dry milk. The dry milk may contain less than 10% milkfat by weight, less than 7% milkfat by weight, less than 2% milkfat by weight, or less than 1% milkfat by weight. In various embodiments, the body scrub composition comprises dry milk which is substantially free of milkfat. In some embodiments, the body scrub composition comprises soy flour made from whole soy beans.

In certain embodiments, the ground whole oats are made from organic oats, which are grown and processed without exposure to chemical contaminants or pesticides. In certain embodiments, the ground whole oats are made from organic oats which have not been grown or processed near wheat, barley, or other grains containing gluten with high levels of prolamines, so as to minimize the risk of an allergic reaction to grain glutens.

Soy flour is naturally gluten-free, and is a suitable substitute for wheat- and corn-based ingredients in conventional products. In certain embodiments, the soy flour is made from organic soybeans which have not been genetically modified.

In various embodiments, the body scrub composition comprises a carrier. The carrier may be a liquid carrier, mixed into the body scrub composition in an amount sufficient to form a liquid or semi-liquid suspension of the solid ingredients, e.g., a dispersion or a paste. The liquid carrier may be an aqueous carrier or a non-aqueous carrier. Suitable non-aqueous carriers include long-chain alcohols and esters, e.g., cetyl alcohol; vegetable oils, e.g., olive oil; and hydrocarbon carriers, e.g., petroleum jelly. Dimethylsiloxane oils may also be used as moisturizing carriers. Non-aqueous carriers possess useful skin moisturizing properties when added to the body scrub composition.

In various embodiments, the body scrub composition comprises a solid carrier. The solid carrier may comprise a solid soap. A body scrub composition comprises a solid soap carrier may be made by mixing a powdered mixture of ground coffee; ground whole oats; dry milk; and soy flour with a molten soap; and cooling the molten soap in a mold to produce a soap bar. In certain embodiments, the body scrub composition does not contain a carrier, and is used as a powdered mixture of ground coffee; ground whole oats; dry milk; and soy flour.

Various exemplary embodiments disclosed herein relate to a body scrub composition, comprising a mixture of ground coffee; ground whole oats; from about 20% to about 40% by weight dry milk; and from about 0.5% by weight to about 5% by weight of soy flour, based on the weight of the coffee, oats, dry milk, and soy flour. The body scrub composition may comprise a mixture of ground whole oats; dry milk; from about 25% to about 50% by weight ground coffee; and from about 0.5% by weight to about 5% by weight of soy flour. Alternatively, the body scrub composition may comprise a mixture of ground coffee; dry milk; from about 25% to about 50% by weight ground whole oats; and from about 0.5% by weight to about 5% by weight of soy flour.

In various embodiments, the body scrub composition comprises from about 25% to about 50%, from about 30% to about 40%, or about 35% by weight ground coffee; from about 25% to about 50%, from about 30% to about 40%, or about 35% by weight ground whole oats; from about 20% to about 40%, from about 25% to about 35%, or from about 28% to about 30% by weight dry milk; and from about 0.5% by weight to about 5%, from about 0.5% to 3%, or about 1% by weight of soy flour; and an optional carrier, where all percentages are based on the combined weight of the coffee, oats, dry milk, and soy flour.

Various exemplary embodiments disclosed herein relate to a method of making a body scrub composition, by combining ground coffee, ground whole oats, dry milk, and from about 0.5% by weight to about 5% by weight of soy flour; and mixing the product of step (a) to produce a uniform powdered mixture. The uniform powdered mixture may be used directly as a body scrub composition, or mixed with a carrier. The carrier may be an aqueous carrier; a non-aqueous liquid carrier, such as a silicone oil, a vegetable oil, or a long chain alcohol or ester; or a solid soap.

Certain embodiments disclosed herein relate to a body scrub composition, comprising ground coffee; ground whole oats; casein; and from about 0.5% by weight to about 5% by weight of soy flour, based on the total weight of the coffee, oats, soy flour, and casein. The body scrub composition may further comprise an optional carrier.

### EXEMPLARY EMBODIMENT

### Example 1

A body scrub formulation is prepared from the following ingredients:

roast coffee (10 tablespoons; 236.45 g);

100% natural oats (16 tablespoons; 236.45 g);

low fat dry milk (13 tablespoons; 192.12 g); and

100% whole bean soy flour (1 teaspoon).

The body scrub formulation is prepared by the following process:

1) Grind 10 tablespoon of dark roasted coffee beans for 60 sec.

2) Grind 16 tablespoons of natural whole grain oats for 2 min until the whole grain oats become a smooth powder, like flour;

3) Combine the ground coffee beans and the ground oats with 13 tablespoons of low fat dry milk and 1 teaspoon whole bean soy flour;

4) Mix the combined dry ingredients very slowly, e.g., in a blender at low speed, until the mixture becomes a smooth, flowable powder.

The smooth flowable powder may be used as a body scrub, or combined with an aqueous carrier or a non-aqueous carrier, e.g., a liquid oil or other emollient.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1. A body scrub composition, comprising:
a) ground coffee;
b) ground whole oats;
c) dry milk;
d) from about 0.5% by weight to about 5% by weight of soy flour, based on the total weight of ingredients (a), (b), (c), and (d); and
e) an optional carrier.

2. The body scrub composition of claim 1, wherein said dry milk comprises less than 10% milkfat by weight.

3. The body scrub composition of claim 2, wherein said dry milk comprises less than 7% milkfat by weight.

4. The body scrub composition of claim 3, wherein said dry milk comprises less than 2% milkfat by weight.

5. The body scrub composition of claim 4, wherein said dry milk comprises less than 1% milkfat by weight.

6. The body scrub composition of claim 1, wherein the carrier is an aqueous carrier.

7. The body scrub composition of claim 1, wherein the carrier comprises at least one vegetable oil.

8. The body scrub composition of claim 1, wherein the carrier comprises a solid soap bar.

9. The body scrub composition of claim 1, wherein the body scrub composition does not contain a carrier.

10. The body scrub composition of claim 1, comprising from about 20% to about 40% by weight dry milk.

11. The body scrub composition of claim 1, comprising from about 25% to about 50% by weight ground coffee.

12. The body scrub composition of claim 1, comprising from about 25% to about 50% by weight ground whole oats.

13. The body scrub composition of claim 1, wherein the soy flour is whole grain soy flour.

14. The body scrub composition of claim 1, comprising:
a) from about 25% to about 50% by weight ground coffee;
b) from about 25% to about 50% by weight ground whole oats;
c) from about 20% to about 40% by weight dry milk;
d) from about 0.5% by weight to about 5% by weight of soy flour; and
e) an optional carrier;
wherein all percentages are based on the combined weight of ingredients (a), (b), (c), and (d).

15. The body scrub composition of claim 14, comprising:
a) from about 30% to about 40% by weight ground coffee;
b) from about 30% to about 40% by weight ground whole oats;
c) from about 25% to about 35% by weight dry milk;
d) from about 0.5% by weight to about 3% by weight of soy flour; and
e) an optional carrier.

16. A method of making a body scrub composition, comprising:
a) combining ground coffee, ground whole oats, dry milk, and from about 0.5% by weight to about 5% by weight of soy flour; and
e) mixing the product of step (a) to produce a uniform powdered mixture.

17. The method of claim 16, further comprising mixing the uniform powdered mixture with a carrier.

18. The method of claim 17, wherein the carrier is selected from the group consisting of an aqueous carrier and a vegetable oil.

19. The method of claim 17, wherein said mixing the uniform powdered mixture with a carrier comprises:
i) mixing the uniform powdered mixture with a molten soap; and
ii) cooling the molten soap in a mold to produce a soap bar.

20. A body scrub composition, comprising:
a) ground coffee;
b) ground whole oats;
c) casein;
d) from about 0.5% by weight to about 5% by weight of soy flour, based on the total weight of ingredients (a), (b), (c), and (d); and
e) an optional carrier.
